(19) United States
(12) Patent Application Publication
(10) Pub. No.: US 2021/0290814 A1
(43) Pub. Date: Sep. 23, 2021

(54) METHODS AND SYSTEMS FOR STERILIZING SPACES OR SURFACES FROM STAND-OFF DISTANCES

(71) Applicant: Alakai Defense Systems, Inc., Largo, FL (US)

(72) Inventors: Robert Douglas Waterbury, Largo, FL (US); Ed Dottery, Largo, FL (US); Timothy Molner, Largo, FL (US); Marshall Scott, Largo, FL (US)

(21) Appl. No.: 17/301,059

(22) Filed: Mar. 23, 2021

Related U.S. Application Data
(60) Provisional application No. 62/993,504, filed on Mar. 23, 2020.

(51) Int. Cl. A61L 9/20

(52) U.S. Cl. CPC A61L 9/20; A61L 2209/20; A61L 2209/16; A61L 2209/12

(57) ABSTRACT

A method, system, and apparatus of sterilizing or disinfecting a space or surface with UV light from a laser that produces UV or other light energy with germicidal effect, at least like UVGI. The light can be controlled in terms of power to be effective for germicidal effect on a given biological organism or agent. In cases where humans might be present and represent a risk of interaction with the laser, techniques are used to remove those risks. The laser can be directed to a single relatively small area, aimed to multiple areas, or scanned to cover larger areas. It allows either human operation at stand-off distances from the relevant spaces, volumes, areas, surfaces (e.g., at least inches and typically feet or tens of feet). It has an indefinite useful operation life.

### CROSS-REFERENCE TO RELATED APPLICATIONS

This application claims the benefit of Provisional Application U.S. Ser. No. 62/993,504 filed on Mar. 23, 2020, all of which is herein incorporated by reference in its entirety.

### I. BACKGROUND OF THE INVENTION

### A. Field of the Invention

The present invention relates to disinfecting spaces, parts of spaces, or areas, including air and surfaces and, in particular, in an automated or semi-automated fashion and from stand-off distances by using controlled lasers. The methods and systems are applicable to a range of biological organisms and agents including bacteria and viruses, including pathogens like the novel corona virus.

### B. Problems in the State of the Art

The current world-wide pandemic illustrates the significant need for a variety of techniques to combat the spread of viruses of the type that are virulent. Present approaches include social distancing of people, personal practices such as hand washing, avoid face-touching, and covered coughing, face masks and other protective gear, and monitoring for symptoms.

Disinfecting is also used. Disinfecting many times uses antimicrobial agents that attempt to inactivate or destroy microorganisms. Typically, in liquid form or infused into a wipe, this requires direct manual application to surfaces, or at least in close proximity to the surface. They are effective at the surfaces. The limitations are obvious. It requires people. It requires manual action and proximity. It is limited to application areas and for limited efficacy time.

In the example of novel corona virus, the virus can be passed between people in the air or via surfaces. Some air disinfectants are available. But these are also typically limited to a person directing the aerosol or spray into a given space, and therefore only effective for that space and for a limited efficacy time.

In all these cases, further limitations exist. They need replenishment of physical disinfectants, whether liquid, wipes, or aerosols. High demand can cause supply shortages. There are economic and efficiency issues with current approaches, especially in a wide-spread or pandemic situation.

There would be great benefits to methods and systems that could automatically or at least semi-automatically sterilize or disinfect air or surfaces without requiring manual application, as well as have a long useful life, cover substantially spaces or areas, and be effective.

It has been known that ultraviolet (UV) light can have sterilizing or germicidal effects. See, e.g., the discussion of ultraviolet germicidal irradiation (UVGI) at Nicholas G. Reed, The History of Ultraviolet Germicidal Irradiation for Air Disinfection, Public Health Reports, January-February 2010, Vol. 125, pgs. 15-27, which is incorporated by reference here. Such techniques typically emit short-wavelength ultraviolet (UV-C) from lamps using fluorescent, incandescent, LED, or arc discharge lamps. The UV light is either omni-directionally flooded into a space or with some type of reflector system which tries to capture and control the source light generally in a direction. Additionally, there is generally one output intensity for a given electrical power input. As such, there are limitations on such approaches including in terms of control, efficacy, and efficiency.

It can be seen, therefore, that a variety of factors exist in trying to remediate spaces or areas in the context of disinfecting for viruses. Some are antagonistic with one another. For example, liquid and aerosol disinfectants are relatively inexpensive. But they are consumed and must be replenished if disinfecting is needed over a large area or long time. They can become scarce. Another example relates to efficiency of use. UVGI can flood a space with germicidal UV light, but there is little ability to adjust or customize the effective application area and power for each application. This creates uncertainty in efficiency, efficacy, and ability to meet demand/goals of each application or use.

Therefore, the inventors have identified problems and deficiencies in the state of the art as well as room for improvement.

### II. SUMMARY OF THE INVENTION

### A. Objects, Features, and Advantages of the Invention

It is a principal object, feature, aspect, and advantage of the present invention to present methods and systems which solve or improve over problems and deficiencies in the state of the art.

Further objects, features, aspects, and advantages of the present invention provide methods and systems as discussed above which:
a. can be customized easily for any number of different applications;
b. can be effective over a range of sizes of spaces and areas, as well as over a range of time frames;
c. can be automated or semi-automated in operation to reduce human involvement and presence, or at least, allow operation from stand-off distances;
d. can be adjusted or varied for given needs or desires;
e. can be effective for disinfecting air and/or surfaces; and/or
f. can be configured for human safety.

### B. Aspects of the Invention

A first aspect of the present invention relates to a method of sterilizing or disinfecting a space or surface with UV light from a laser that produces UV or other light energy with germicidal effect, at least like UVGI. The light can be controlled in terms of power to be effective for germicidal effect on a given biological organism or agent. In cases where humans might be present and represent a risk of interaction with the laser, techniques are used to remove those risks. The laser can be directed to a single relatively small area, aimed to multiple areas, or scanned to cover larger areas. It allows either human operation at stand-off distances from the relevant spaces, volumes, areas, surfaces, (e.g. at least inches and typically feet or tens of feet). It has an indefinite useful operation life.

Another aspect of the invention relates to a system that allows practice of the method above. A UV laser can have control components to allow selection of power, average power, and the like relative to a space or surface for effective germicidal effect. It can also have control components regarding direction of the laser energy. This could be via manual human aiming or electro-mechanical aiming. The latter could include automated or semi-automated scanning.

Another aspect of the invention relates to the ability to configure a system to fit any of a number of needs or desires. Current UVGI techniques rely on relatively inexpensive mercury arc lamps or LED arrays to generate the UV light. Use of a laser as the UV source creates both challenges but also benefits different from such lamps.

These and other objects, features, aspects, and advantages of the present invention will become more apparent with reference to the accompanying specification.

### III. BRIEF DESCRIPTION OF THE DRAWINGS

Illustrations that assist an understanding of aspects of the invention are included and cited in the exemplary embodiment descriptions that follow.

FIG. 1A is a highly diagrammatic generalized embodiment of an apparatus and system according to aspects of the invention showing use of a laser system to scan free space for germicidal effect.

FIG. 1B is similar to FIG. 1A but installed in a room.

FIG. 1C is a generalized method according to aspects of the invention.

FIG. 2 is a diagrammatic view of a specific exemplary embodiment according to the invention where in the laser is controlled to upper room operation relative to a room that can include presence of people for safety reasons.

FIG. 3 is a diagram showing one exemplary embodiment of laser system and components that can be used in the embodiment of FIG. 2.

FIG. 4 is a diagrammatic illustration of functions and functional components that can be used with embodiments including those of FIGS. 1A-C,-2, and 3.

FIG. 5 is an illustration of application of an embodiment such as FIGS. 2-4 to a large room with many people.

FIGS. 6A and B are information supporting germicidal efficacy of UV light and upper room operation.

FIG. 7 is a top plan view diagrammatic view according to aspects of the invention, here illustrating scanning of a room with a laser.

FIG. 8 is an illustration of application of principles of the embodiment of FIG. 1A or 2 to a large public building.

FIG. 9 is a perspective view of a small form factor UV laser that could be used in embodiments of the invention to produce UVGI energy.

FIG. 10 is a perspective view of another exemplary embodiment according to aspects of the invention; here a laser system such as FIG. 1A is installed at a HVAC vent.

FIGS. 11 and 12 illustrate optional add-on features that could be used with the embodiment of FIG. 6A.

FIG. 13 is a still further exemplary embodiment according to aspects of the invention; here a laser system in a portable hand-held size unit for selective disinfecting of targeted spaces, areas, objects, or surfaces by an operator.

FIG. 14 is another exemplary embodiment according to aspects of the invention, here a robotic vehicle carrying a laser system for disinfecting targeted spaces, areas, objects, or surfaces.

FIG. 15 is a diagrammatic view of an optional feature according to aspects of the invention, namely, using light blocking elements to block laser light from reaching certain space(s) or surface(s) when a laser is scanned.

FIG. 16 is a diagrammatic view of an optional feature according to aspects of the invention, namely, using special materials or surfaces relative to laser light, including laser absorbing material to prevent the laser light from reflecting, laser reflecting material or components to redirect laser light, or patterned, colored, or otherwise distinguishable material or components delimiting the boundary of the laser, and recognizable by camera or other sensors that can correlate location in camera space to location/direction of laser in real physical XYZ space.

FIG. 17 is similar to FIG. 15 but illustrates boundary material that is distinguishable from surrounding surfaces or materials in camera space of a digital camera with image recognition software to control or delimit direction of the laser.

FIGS. 18A-C are diagrammatic illustrations of an optional feature that can be used in embodiments of the invention, where optical techniques spread or fan out the more collimated typical laser beam to cover more 3D space.

### IV. DETAILED DESCRIPTION OF EXEMPLARY EMBODIMENTS ACCORDING TO THE INVENTION

### A. Overview

For a better understanding of aspects of the invention, several examples or exemplary embodiments will now be described. It is to be understood they are neither exclusive nor inclusive of all forms and embodiments the invention can take. For example, variations obvious to those skilled in this technical field are included with the invention.

### B. Generalized Embodiment

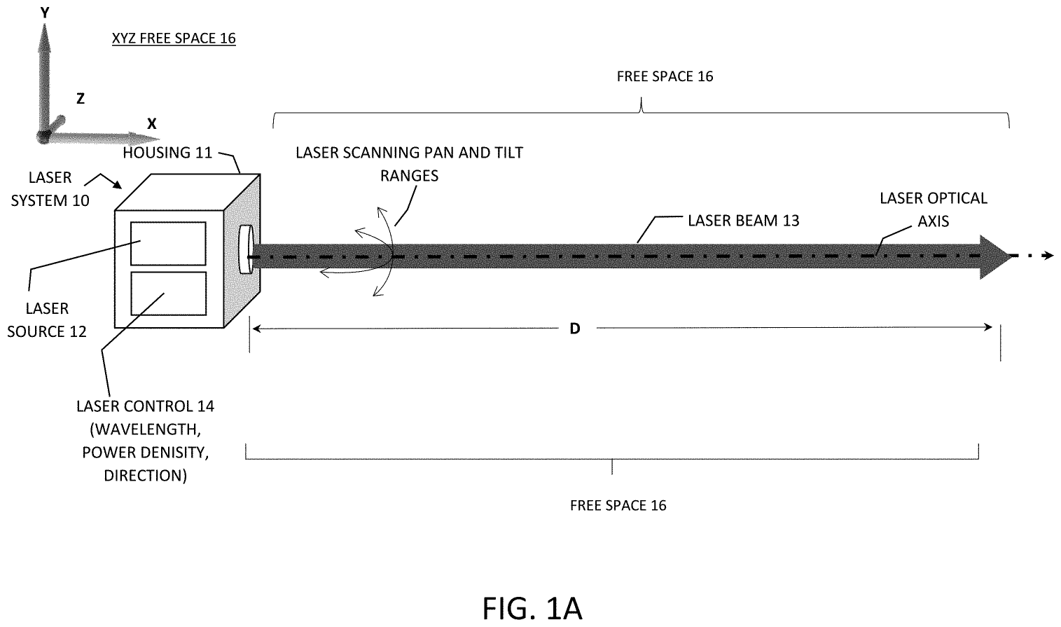

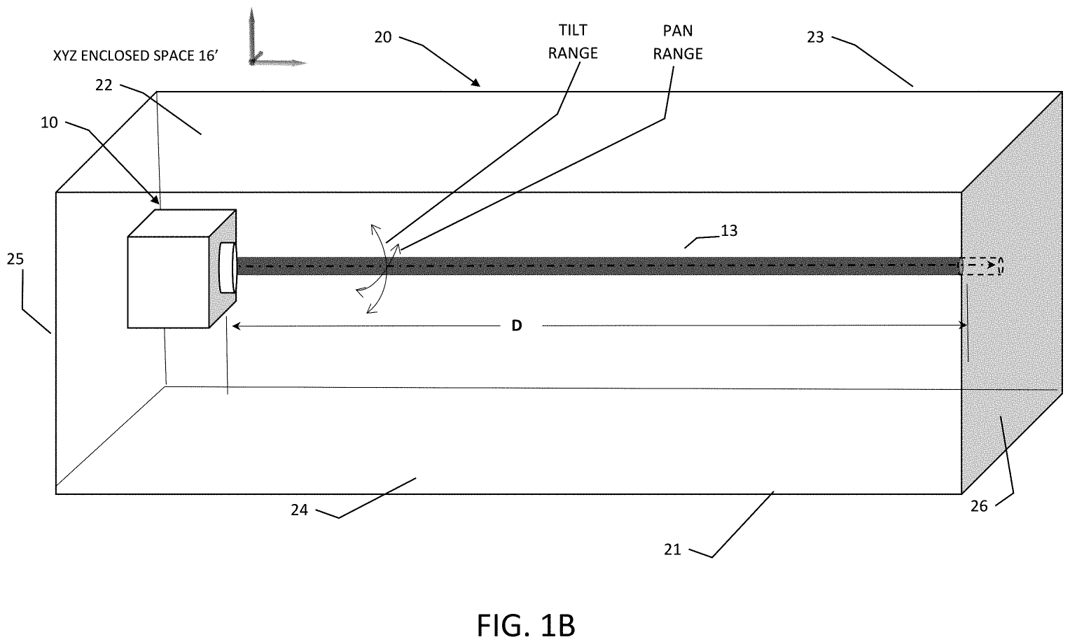

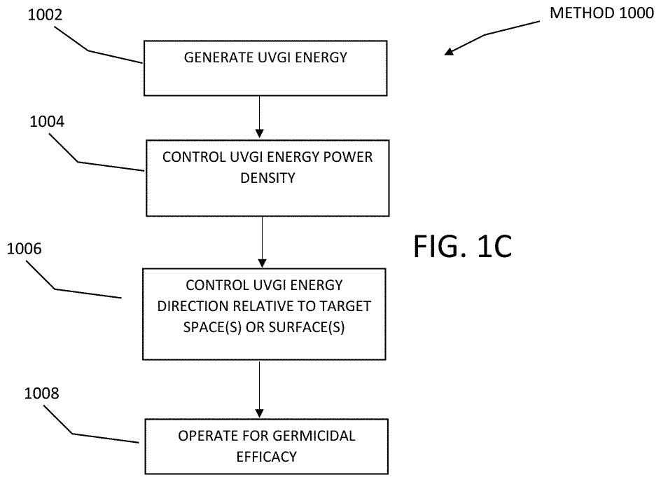

Below are several specific exemplary embodiments. They provide non-limiting examples of specific application of aspects of the invention. As will be appreciated, as indicated in the Summary of the Invention and the Claims, a unifying concept in at least most of the embodiments is the utilization of light energy, one example being UVGI by generating and controlling a laser effective for UVGI. As illustrated in FIGS. 1A-C, a generalized embodiment of the invention does exactly that. It addresses technical problems and deficiencies with a technical solution that provides flexibility, germicidal effectiveness, cost-effectiveness, and other benefits.

A generalized apparatus according to this embodiment includes a laser system 10. A housing 11 can contain all or many of the components of system 10. Housing 11 can be of a variety of sizes but, at least in some embodiments, can be relatively small in form factor (e.g., no more than a few inches in longest dimensions) which can increase flexibility of use and applications).

System 10 includes a light source 12 that produces light energy 13 with germicidal effect. One example is UVGI produces by a laser. The designer can select the wavelength, power density, beam size and pattern, and other operating parameters according to need or desire for a given application. In one form, the laser produces UVGI in a substantially collimated beam along a reference or home optical axis, and at a power density of sufficient strength for germicidal efficacy according to published reports. The type, nature, and operating characteristics are one form of control 14 of the germicidal light of system 10.

Another form of control 14 in system 10 is direction control of the light energy. As indicated diagrammatically in FIG. 1A, system 10 include components and/or techniques to change the direction and/or direction D of the light energy 13 produced by light source 12. Examples of such components and techniques are known to those skilled in the art. For example, a collimated laser 13 from a fixed location laser source 12 can be altered in directionality in various ways to change direction from a home or reference direction. One well-known example is electro-mechanical techniques to alter the beam direction such as laser scanners embedded into checkout counters which scan products for bar codes. Such techniques can essentially tilt and pan the beam from reference position over ranges that allow 2D and 3D scanning of an XYZ free space 16 (i.e., not necessarily enclosed in any direction) or XY, YZ, or XZ area much larger than the beam width (see, e.g., FIG. 1A), but also an enclosed or partially enclosed space 16′ like a room 20 (see, e.g., FIG. 1B). As such, these techniques can be implemented in system 10 to provide scanning ability in 3D free space 16 over scan tilt and pan ranges (e.g. relative to a home or refence optical axis for the beam), including those that could cover at least most of a room in a building. As will be further appreciated by those skilled in the art, substantially collimated lasers can carry energy over long distances will maintaining power density minimums. As such, system 10 benefits from that ability. System 10 can service a small XYZ space (a few cubic feet) to large XYZ spaces (hundreds and even thousands of cubic feet).

The controllability of a laser (e.g. power, wavelength, direction) allows placing germicidal/sterilizing/disinfecting UV energy to spaces and areas from what will be called stand-off distances even if the laser is operated by a human. Stand-off distance is a well-known term in the military. It can be inches to feet to tens of feet to hundreds of feet, and sometimes more. It is stand-off in the sense it does not require contact with the relevant space or area except for interrogation by the light energy in the laser beam, and importantly, it can be from quite far away, which in terms of pathogens (e.g., highly communicable viruses), can be important for the safety of humans.

As further indicated, implementation of UVGI by laser is more than simple “illuminating” a surface, space, or area. Aspects of the invention provide technological solutions to competing factors and interests involved in use of lasers for such purposed, including laser that produce UVGI. For example, the presence of lasers can present human safety issues, including eye safety and skin safety. Aspects of the invention can include techniques to deal with such issues. Thus, the generalized embodiments meet or exceed at least some of the objects, features, aspects, or advantages of the present invention. A method 1000 according to the invention does likewise.

Method 1000 generates UVGI energy (step 1002). The designer selects the energy source to control the type, power density, distribution pattern, and other operating parameters for a given application (step 1004). The selected light energy is controlled directionally to service a desired XYZ space 16 or 16′ and/or sub-spaces, sub-areas, or sub-surfaces of the same (step 1006). Method 1000 combines these steps for germicidal efficacy for a given application (step 1008).

As such, system 10 and method 1000 allow a designer high flexibility and options for each application. The stand-off distance for the light source and its ability to service relatively large XYZ spaces from stand-off distances is advantageous both for operators and users of the XYZ space.

Further understanding of the aspects of the invention will be seen from the specific embodiments described below.

### C. Specific Exemplary Embodiments

Below are several specific exemplary embodiments. These embodiments implement at least the basic concepts of the generalized embodiments discussed above.

### UV Germicidal Irradiation Using a UV Laser

Ultraviolet Germicidal Irradiation (UVGI) is a proven technology that has been around for decades. The following references, each incorporated by reference in its entirety, give a good history:
a. DHHS Centers for Disease Control and Prevention (NIOSH) Publication No. 2009-105.
b. Reed N G. The history of ultraviolet germicidal irradiation for air disinfection. Public Health Rep. 2010; 125(1):15-27. doi:10.1177/003335491012500105. This 2010 report from the U.S. Army Center for Health Promotion and Preventive Medicine states: “Ultraviolet Germicidal Irradiation (UVGI) is an established means of disinfection and can be used to prevent the spread of certain infectious diseases”. This report also states that “if used properly, UVGI can be safe and highly effective in disinfecting the air, thereby preventing transmission of a variety of airborne infections.”
c. Kowalski, Wladyslaw. Ultraviolet germicidal irradiation handbook: UVGI for air and surface disinfection. Springer science & business media, 2010.
d. Escombe A R, Moore D A J, Gilman R H, Navincopa M, Ticona E, Mitchell B, et al., Upper-room ultraviolet light and negative air ionization to prevent tuberculosis transmission. PLoS Med 2009; 6:e43.
e. https://www.genetex.com/Research/Overview/infectious_diseases/SARS-CoV-2.
f. Tseng, Chun-Chieh, and Chih-Shan Li. “Inactivation of virus-containing aerosols by ultraviolet germicidal irradiation.” Aerosol Science and Technology 39.12 (2005): 1136-1142.

The technology is mature enough that the United States Center for Disease Control (CDC) published a report in 2009 titled: Environmental Control for Tuberculosis: Basic Upper-Room UVGI Guidelines for Healthcare Settings, PHHS (NIOSH) Publication No. 2009-105, March 2009 available on-line on Mar. 22, 2021 at https://www.cdc.gov/niosh/docs/2009-105/default.html, incorporated by reference herein.

Historically UVGI has been implemented using UV lamps. The embodiments herein all involve the use of UV lasers, and the implementations are allowed by lasers. Below is a detailed description of different non-limiting exemplary implementations.

In all cases described below a UV laser in the wavelength range of 247-280 nm could be utilized as a source. The invention is not necessarily limited to this specific spectra.

### 1. Example Embodiment 1: Upper-Room UVGI

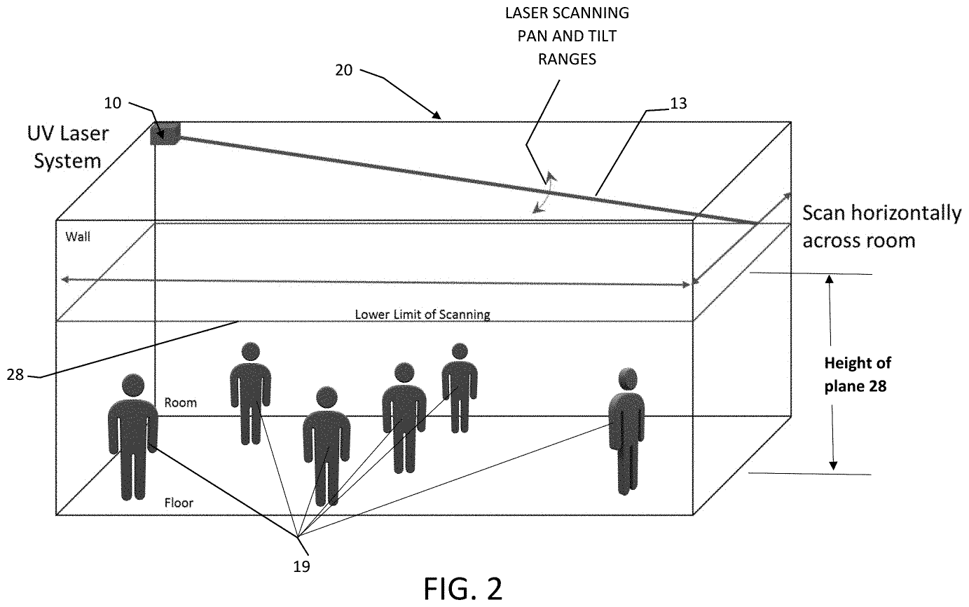

A first specific embodiment according to the invention utilizes apparatus and methods as above-described in the specific context of rooms or spaces where people may be present. With particular reference to FIG. 2, a laser system 10 is mounted inside a room 20. System 10 is configured to scan the upper XYZ space of room 20 above plane 28, which is above the range of typical human height. As such, persons 19, whether standing or sitting in room 20, would never be exposed to direct line-of-sight of laser beam 13. This effectively makes operation of UV laser system 10 eyesafe. Persons 19 can be present in room 20 and germicidal effect of system 10 operated on a continuous basis.

Limiting laser operation to the upper-room for eye safety (and skin safety) would seem counter-intuitive to deterring germ transmission between people in the lower room. However, as discussed below, the combination of features of the invention provides a technical solution to what seems irreconcilable.

The Reed Public Health Rep. 2010; 125(1):15-27 report, cited above, provides a good history of UVGI for air disinfection application and admits that there have been a number of attempts at this over the past 100 years that show efficacy in testing environments but lack success due to implementation issues. This embodiment of the invention addresses these issues.

First, the embodiment uses small, less expensive UV lasers to solve some of the implementation issues thereby allowing new implementation approaches to an existing and proven air disinfection technique. Specifically, this embodiment uses variations of Upper-Room UVGI in which the UV source 12 is active in the room sterilizing airborne viruses and bacteria, while occupants 19 may or may not be in the room. This is shown in FIG. 2. An editor's note from the Escombe et al. study (cited herein) on this technique, says “the UV light approach might provide a relatively low-cost intervention for possible use in waiting rooms and other overcrowded settings where patients with undiagnosed, untreated tuberculosis-individuals who tend to be highly infectious—are likely to come into contact with other susceptible patients, health care workers, and visitors”.

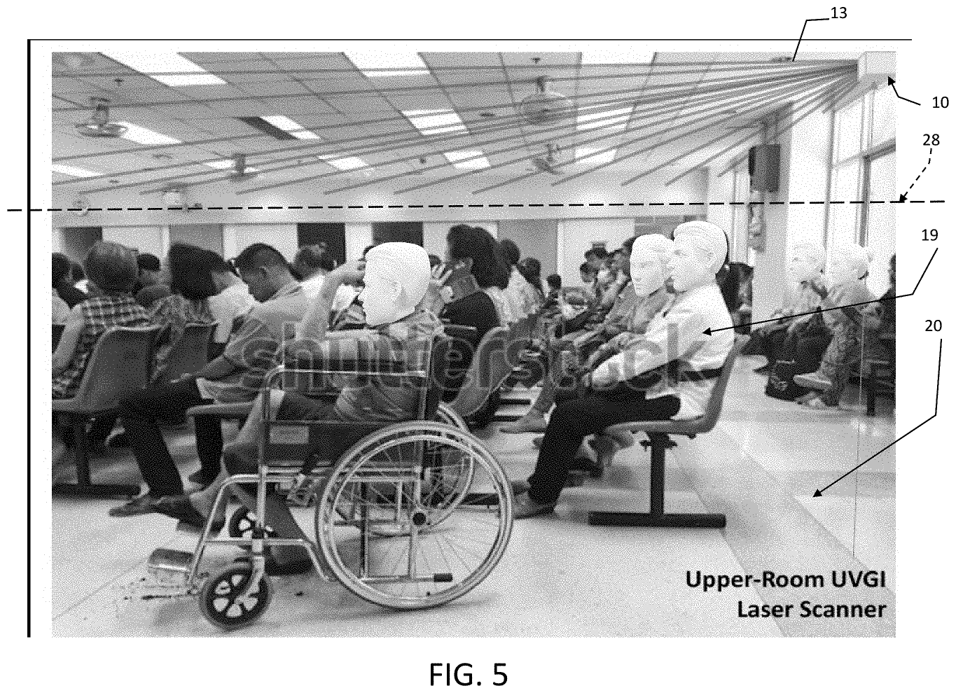

The concept diagram in FIG. 5 shows an Upper-Room UVGI system using a new UV Laser Scanning technology 10 in a crowded hospital waiting room. Reported results using older technology has shown effectiveness in tests at disinfecting airborne bacteria or viruses thereby preventing the cross contamination of personnel.

The embodiment of FIGS. 2-5 provides a practical technical solution. If Upper-Room UVGI could be effectively deployed in crowded areas where there may be airborne virus or bacteria (e.g., COVID-19) and possibly infected people, then it may reduce the probability of infecting additional people. The ways the embodiment of FIGS. 2-5 addresses some of the implementation problems which have prevented widespread use in the past.

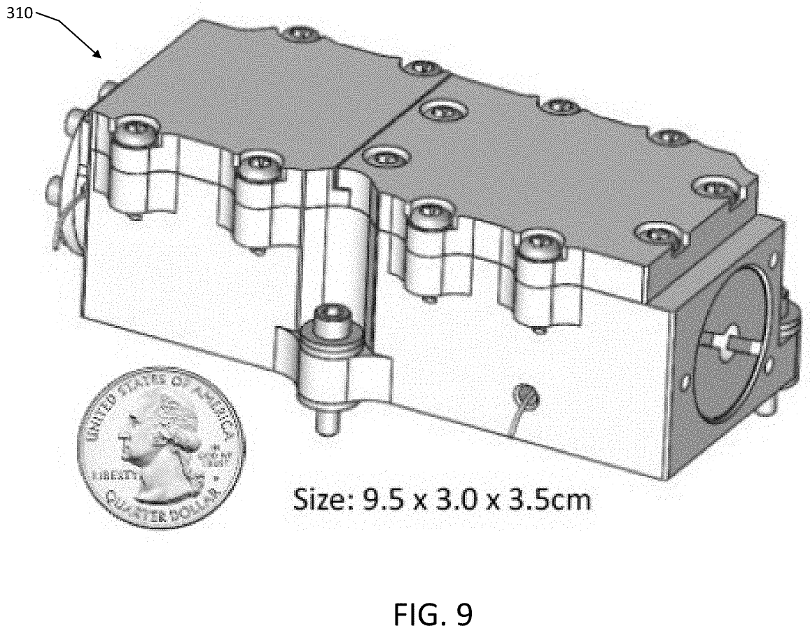

The technology of FIGS. 2-5 enables these new implementations as follows:
a. A small UV Laser 310 (e.g. FIG. 9) developed by the present Applicant for use in a miniaturized DARPA sensor system is utilized here.
b. The Applicant's Patented Stimulated Aversion technology ensures eye safety when/if proximity to humans is desired. See U.S. Pat. No. 8,724,097, incorporated by reference herein.
c. Optionally, Artificial Intelligence (AI) can be used to automate and control the overall system, eventually eliminating operators. By AI it is meant that by machine-learning or neural network type automated or semi-automated operation via hardware and software, the system 10 can learn how best to service a given XYZ space with germicidal UVGI. Those skilled in the art understand how a system can be programmed to do so.

### UVGI Air Disinfection—Key Info

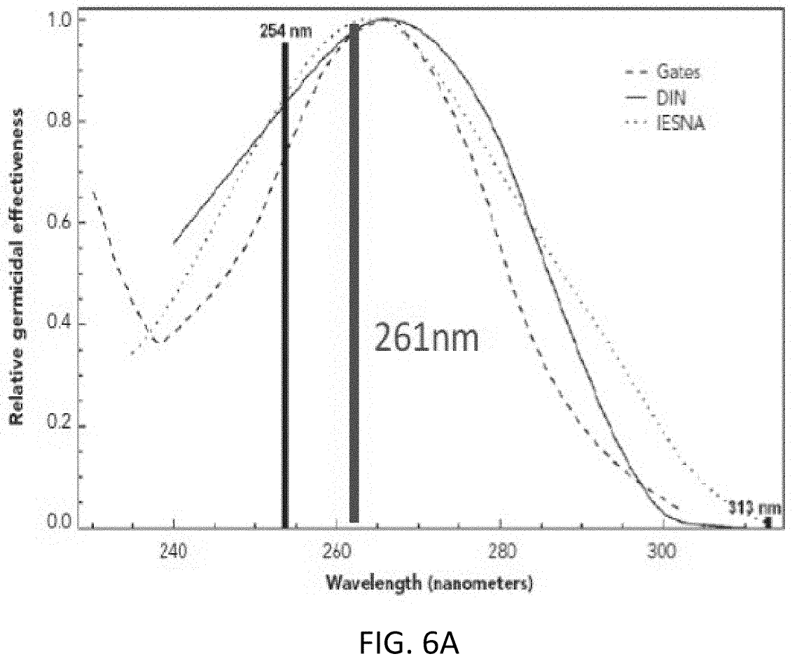

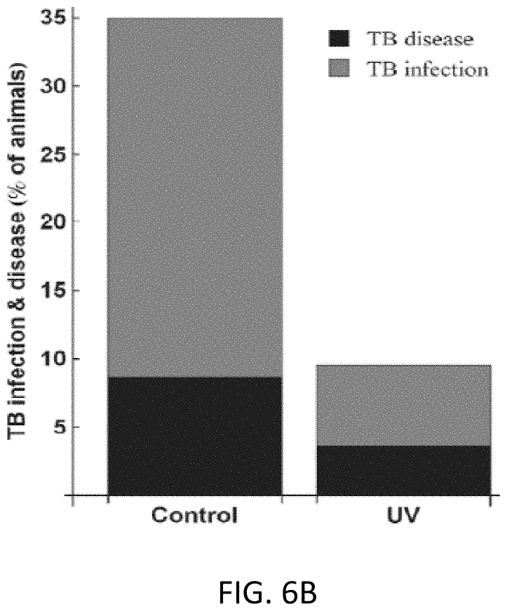

a. UVGI has been around since the late 1800's and for the last 75+ years the most common UV source has been a Mercury lamp since it has an emission wavelength which is close to the peak absorption wavelength of common and bacteria (see FIG. 6A). This Figure also shows the operating wavelength of the Applicant's laser (261 nm) of FIG. 9 which is slightly closer to the absorbance peak thereby increasing the effectiveness of UVGI with our proposed source.
b. A recent study investigated the effectiveness of Upper-Room UVGI Air Disinfection on guinea pigs who were subjected to Tuberculosis (TB) disease [Escombe, et al. cited herein]. This was a double-blind study with a control group that showed the TB infection rate and TB disease rate were significantly reduced for the animals in locations with Upper-Room UVGI (see FIG. 6B) [Reed, cited herein].

### UVGI—Two Approaches

a. UVGI Air Disinfection can be broken down into two main groups; (1) Room based UVGI and (2) In-ductor Air handler based UVGI. This paper only deals with Room-based UVGI since it has a more effective impact on preventing the spread of airborne viruses (e.g., COVID-19) and bacterial than the other approach [Reed, cited herein]. Guidelines for implementing effective Upper-Room UVGI are described in the CDC report [DHHS Centers for Disease Control and Prevention (NIOSH) Publication No. 2009-105, incorporated by reference herein].

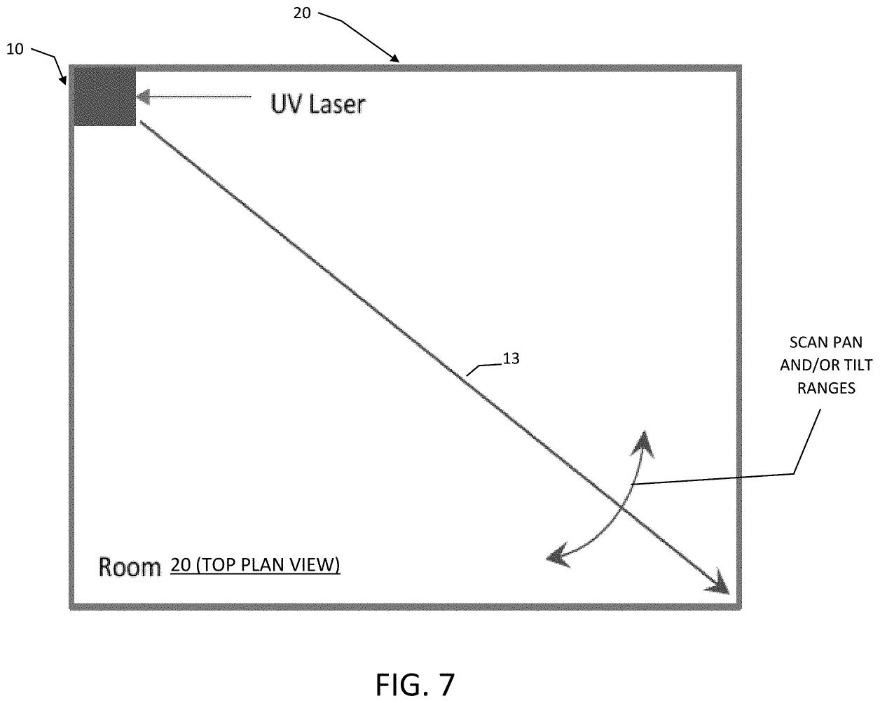

FIG. 6A shows a comparison of the absorbance spectra of bacteria from three different studies to the peak emission wavelength for lamps (254 nm) and the new Laser (261 nm). FIG. 6B shows a comparison of the TB infection rate and disease rate between animals with Upper-Room UVGI and a control group [see Reed reference cited herein]. FIG. 7 illustrates the concept of the new Upper Room UVGI Air Disinfection approach using a scanning UV laser.

The lasers are the most efficient way to send the germicidal energy using a scanning UV laser. Below contains descriptions of the justification for the Upper-Room UVGI system of FIGS. 2-9. There are two different implementations which are described below. All of the eye safety and other implementations discussed in the following sections apply to both implementations.

a. Method for Scanning the UV Laser

i) Laser Scanning System

A diagram of the laser scanner system is shown in FIG. 2. The laser scanner optionally may be programmed to have a slight wobble to it (1″-6″) so that does not hit the same place on the opposite wall thereby minimizing the UV discoloration of the paint or covering on the wall of a room, if it impacts such a surface.

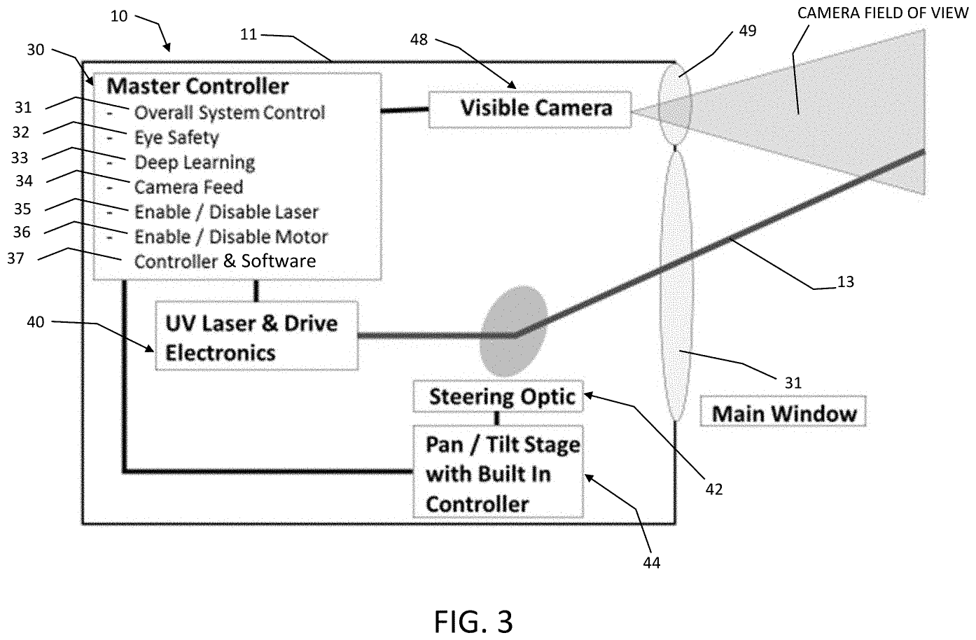

One example of a scanning laser and control system is shown in FIG. 3. As can be seen, and as will be appreciated by those skilled in the art, system 10 can include in housing 11 at least the following components.

Master controller 30.

Any single board computer can serve this function (an example is an ODROID-XU4 board from https://wiki.odroid.com/odroid-xu4/odroid-xu4 available commercially from, e.g., ameriDroid at 245 E. Perkins, St. Ukiah, Calif. 95482 (USA) (www.americroid.com).

An example of one way to configure the specifications of controller board 30 is as follows:

| | |
|---|---|
| Processor | Samsung Exynos5 Octa ARM Cortex ™-A15 |
| | Quad 2 Ghz and Cortex ™-A7 Quad 1.3 GHz |
| | CPUs |
| Memory | 2 Gbyte LPDDR3 RAM at 933 MHz (14.9 |
| | GB/s memory bandwidth) PoP stacked |
| 3D Accelerator | Mali-T628 MP6(OpenGL ES 3.0/2.0/1.1 and |
| | OpenCL 1.1 Full profile) |
| Video | supports 1080p via HDMI cable(H.264 + AAC |
| | based MP4 container format) |
| Video Out | Standard Type A HDMI connector |
| Audio | Instead of On-board Audio codec, There is I2S |
| | Expansion Port(CON11) |
| USB3.0 Host | SuperSpeed USB standard A type connector × |
| | 2 port, Max Load: total 2 Amp for two USB |
| | 3.0 host ports |
| USB2.0 Host | High Speed standard A type connector × 1 |
| | ports, Max Load: 500 mA/port |
| Display | HDMI monitor |
| Storage (Option) | MicroSD Card Slot, eMMC module socket: |
| | eMMC 5.0 HS4000 Flash Storage |
| Gigabit Ethernet LAN | 10/100/1000 Mbps Ethernet with RJ-45 Jack |
| | (Auto-MDIX support) |
| Serial console port | Connecting to a PC gives access to the Linux |
| | console. You can see the log of the boot, or |
| | to log in to the C1 to change the video or |
| | network settings. Note that this serial |
| | UART uses a 1.8 volt interface. We |
| | recommend the USB-UART module kit from |
| | Hardkernel. Molex 5268-04a(2.5 mm pitch) |
| | is mounted on the PCB. Its mate is |
| | Molex 50-37-5043 Wire-to-Board Crimp |
| | Housing. |
| RTC (Real Time Clock) | If you want to add a RTC functions for logging |
| backup battery | or keeping time when offline, just connect |
| connector | a Lithium coin backup battery (CR2032 or |
| | equivalent). All of the RTC circuits are |
| | included on the ODROID-XU4 by default. |
| | Molex 53398-0271 1.25 mm pitch Header, |
| | Surface Mount, Vertical type (Mate with |
| | Molex 51021-0200) |
| WiFi (Option) | USB WIFI Modules |
| HDD/SSD SATA | SuperSpeed USB (USB 3.0) to Serial |
| interface (Optional) | ATA3 adapter for 2.5″/3.5″ |
| | HDD and SSD storage |
| Power (Option) | 5V 4A Power |
| Case(Option) | Mechanical case & cooler (90 × |
| | 59 × 28 mm approx.) |
| PCB Size | 83 × 58 × 20 mm approx. |

Controller programming and operation is within the skill of those skilled in the art and an operating manual can be found at wiki.odroid.com/odroid-xu4/odroid_xu4, available on-line on Mar. 23, 2021.

As indicated in FIG. 3, it can be configured for a variety of functions, some of which are optional. Examples are:

Overall system control 31. Controller 30 would operate the laser source 12 and directionality.

Eye safety 32. Controller 30 could be set up to delimit the direction of the laser while scanning to areas where human eyes will not be.

Deep learning 33. By ways well-known to those skilled in the art, algorithms can be programmed or accessed to train the scanning of the beam according to a variety of factors.

Camera feed 34. Optionally, a digital camera or imager 48 can have a field of view covering a designated space or area relative the scanning beam. By ways appreciated by those skilled in the art, image processing software can add functionalities. One would be to detect presence of an object in the field of view indicative of a human, and issue that can be used by the controller to shut off the laser (or otherwise avoid that space). Another would be analysis of the images in the field of view of the camera for image recognition. One example is to know limits of laser scanning (e.g. a perimeter, plane, specific areas) where the processor is informed from the camera images and recognition to not direct the laser.

Details of types and operation of presence sensors, including camera-based, to detect presence of objects of interest including humans, can be found at U.S. Pat. No. 8,963,088 issued Feb. 24, 2015 to Barlow et al. entitled Passive infrared range, size, and direction finding proximity detector, incorporated by reference herein; and U.S. Pat. No. 9,516,022B2 issued Dec. 6, 2016 to Borzycki et al. entitled Automated meeting room, incorporated by reference herein.

Enable/Disable Laser 35. As mentioned, the controller, or the controller in combination with the camera (or one or more other sensors), could automatically shut off the laser if the camera or sensor measure or detect something indicative of need to shut off the laser. For example, instead of a camera, a presence sensor (sometimes called proximity sensor) could sense presence of an object indicative of a human and generate a signal to shut off the laser.

Enable/disable motor 36. In embodiments where electric motors are used such as to scan the beam, similarly a camera or other sensor could generate a signal that the controller decides should stop scanning, or at least scanning in one or more directions or areas.

Controller/software 37. Those skilled in the art understand how to set up and program controllers such as an ODROID controller discussed herein, including how to operate laser scanning and perform image recognition with an appropriate digital camera operatively connected.

UV Laser and Drive Electronics 40, and Scanning Components.

As illustrated in FIG. 3, one example of a laser scanning set up would utilize electro-mechanical devices that can be instructed electronically to precisely, accurately, and repeatedly scan a given space or area for a given beam width.

A non-limiting example of a laser driver 40/44 and its operating characteristics can be found at Document Number: Compact-506S-9021 from ScannerMax regarding the specifications of Compact 506 Optical Scanner, Revision 4 Jan. 2020 available on-line on Mar. 22, 2021 at www.scannerMAX.com, which is incorporated by reference herein.

A non-limiting example of a scanner optical mirror 42 and its operating characteristics can be found at Document Number: MACH-DSP_1 from ScannerMax regarding the specifications of Mach-DSP Servo Driver for all ScannerMAX galvos, Revision 4 Jan. 2020 available on-line on Mar. 22, 2021 at www.scannerMAX.com, which is incorporated by reference herein.

Details of optical beam steering and control can be found at US20170361398A published Dec. 21, 2017 to Kleinert entitled Phased array steering for laser beam positioning systems, incorporated by reference herein; and US2003/0222143 issued Dec. 4, 2003 to Mitchell entitled PRECISION LASER SCAN HEAD, incorporated by reference herein.

Visible Camera 48.

As mentioned, a digital camera or imager can be used as a sensor for a variety of purposes.

A non-limiting example of a digital camera and its operating characteristics can be found at e-CAM51_USB Datasheet Revision 1.5, Monday, Aug. 27, 2012 from e-con Systems India Pvt Ltd, 17, 54^{th} St., Ashok Nagar, Chennai-600083 (India), downloadable Mar. 22, 2021 at www.e-consystems.com, which is incorporated by reference herein.

As will be appreciated, housing 11 could include a main window 31 for egress of the laser beam without significant losses. It can also include a window 49 for camera 48 and its field of view. Housing 11 can be at least substantially enclosed and even substantially sealed to resist penetration of water, moisture, dust, or debris.

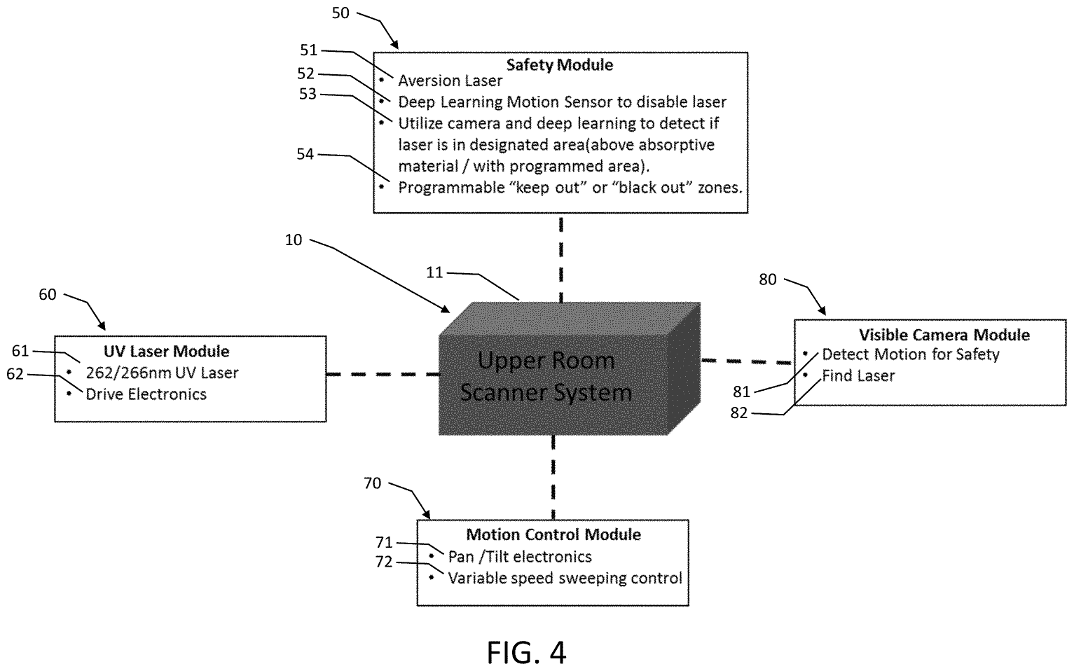

A non-limiting example of a flow chart for the safety subsystems useable with the system 10 is shown in FIG. 4.

As indicated in FIG. 4, an upper-room system according to the invention can include one or more of these functions. Appropriate programming of the scanning directions and limits on the same can be configured according to need or desire for a given application. Software resident on or accessible by controller 40 and camera 48 can be programmed for such functions.

Required UV Energy/Fluence

An initial review of several UVGI studies shows that the required UV radiation level (fluence) to kill airborne bacteria and viruses ranges from 50 uJ/cm² [CDC Publication 2009-105 cited above] to 423 μJ/cm² [TS01]. The higher number of 423 uJ/cm² is specific to a single stranded RNA (ssRNA) virus which is of the same classification as COVID-19 [https://www.genetex.com/Research/Overview/infectious_diseases/SARS-CoV-2 cited above]. The small UV laser of FIG. 9 produces 100 μW of 261 nm light in a 5 mm diameter beam of 509 mW/cm² power density. Thus, at this power level it will only take the beam 2 milliseconds (ms) of time for the UV beam to sterilize airborne virus particles. If we assume a room which is 50×50 ft (15×15 m), then the beam could perform a single sweep of the entire upper room in 6.6 s thereby sterilizing any airborne particles which were in its path (see FIG. 7). The UV laser would continue to scan back and forth, and as new airborne particles were swept into the beam by the AC system they would be sterilized. If the room was 10 ft high (3 m) then the scanner would scan a volume equivalent to the entire room in 1.1 hours. This process can continue 24 hrs/7 days a week even while people are in the room since only the Upper-Room air (volume which is ^{˜}6″ from the ceiling and the people's heads) is sterilized.

ii) Fan-Out of the UV Laser to Cover an Area.

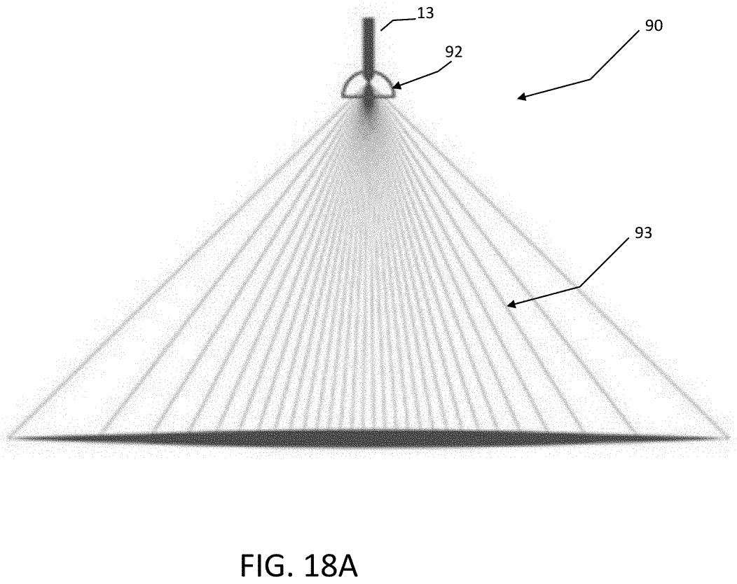

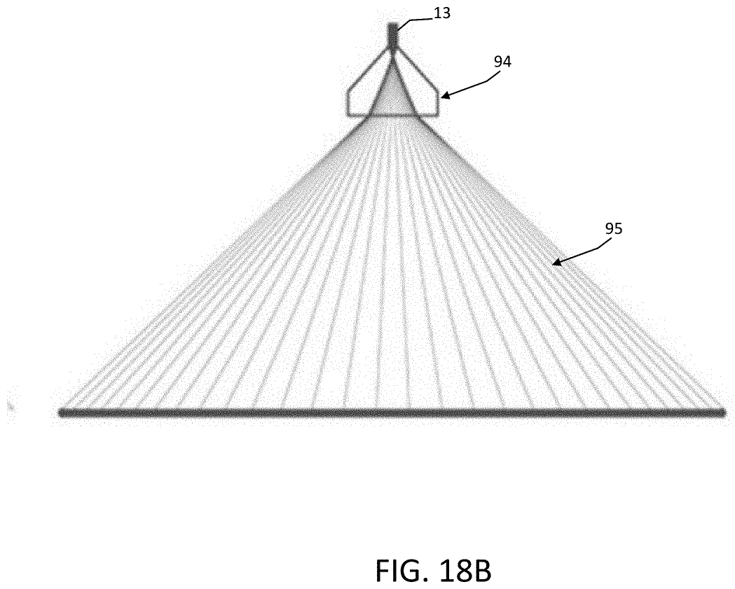

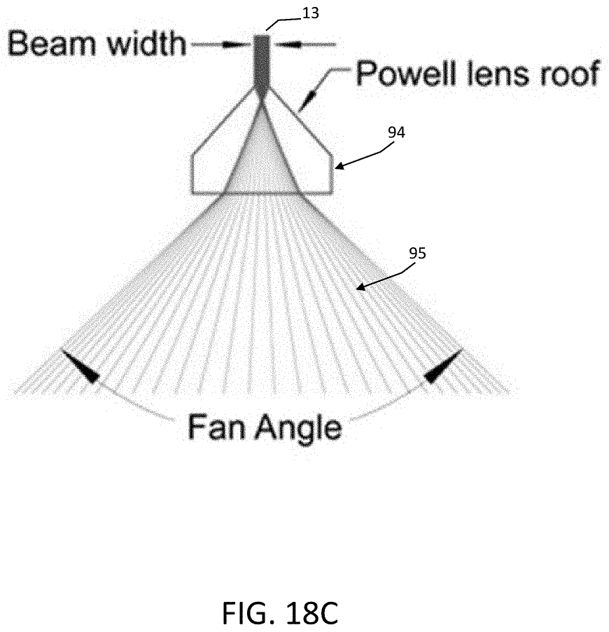

With this concept a series of optical elements are utilized to spread out the optical power of the UV laser beam to cover the desired area. The optical elements would either use refraction or reflection to do this. The refraction technique is shown in FIGS. 18A-C.

Fan Angle

The width of the Powell lens fan angle is a function of the refractive index of the glass and the roof angle of the lens. The steeper the roof and the higher the refractive index, the wider the fan angle and the longer the line for a given projection distance. Small fan angles typically use optical glass of a moderate, e.g., n=1.5, refractive index. Large fan angles are best made with a high index glass, e.g., n=1.8, or higher to minimize the roof's otherwise steep angle.

By comparing set-up 90 with domed lens 92 (FIG. 18A), and a Powell lens 94 (FIGS. 18B and C), it can be seen how the Powell lens 94 distributes energy 95 compared to 93 more evenly in XYZ space. Either lens is possible as are other ways to manipulate or spread the laser beam.

Details about how Powell lenses work, including with lasers, can be found at U.S. Ser. No. 10/401,617B2 issued Sep. 3, 2019 to Hargis, et al. entitled Laser systems and optical devices for manipulating laser beams incorporated by reference herein, and U.S. Pat. No. 9,784,957B2 issued Oct. 10, 2017 to Nackrud entitled Uniformity adjustment method for a diode-laser line-projector incorporated by reference herein.

b. Safety Related Sub-Systems 50

A laser-based system can benefit from, and may be required by law or regulation, to have additional sub-systems which insure they are safe to the public. Below are several safety-related sub-systems which have been developed for and can be used for these implementations.

(1) Stimulated Aversion Eye-Safety Technique 51. Alakai U.S. Pat. No. 8,724,097 at which is Incorporated by Reference Here.

(2) Lock Out Areas

Utilizing a camera placed in the UV scanner box and Artificial Intelligence (AI) and facial recognition routines 52, 53, and 54, the scanner could shut off the UV laser if any faces got close to the area being scanned by the UV laser.

(3) Physical Beam Blocks

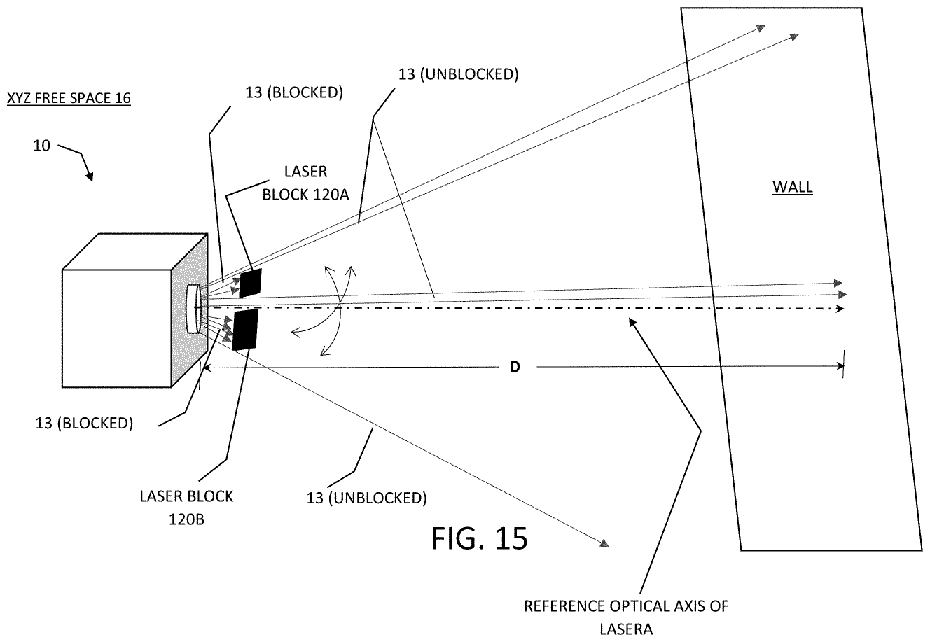

If there are areas that users want to insure have absolutely no UV radiation transmitted down range, it is possible to include physical beam blocks at the appropriate place on the output aperture. See, e.g., diagram of FIG. 15. The nature and shape of blocks 120A and B can be designed to block the scanning from certain directions. Those shapes and position in the scanning range of the laser can be according to need or desire. This approach is the safest since it is based on first principles and works even when software and control electronics sub-systems may fail. FIG. 4 illustrates other possible system functions. They can include but are limited to one or more of the following.

UV Laser Module 60. One example of selectable parameters and functioning is UV wavelengths 61. Others are possible. Drive electronics 62 can be configured as needed.

Motion Control Module 70. Pan/tilt 71 ranges for laser directionality can be configured as needed or desired.

Visible Camera Module 80. Non-limiting examples of functions include some type of sensing including sensing for presence of humans 81 to turn the laser off or divert from sensed humans. Another is variable speed scanning 72. Others are, of course, possible.

(4) Eliminating Reflections

Two approaches are presented for this.

(a) Special Absorbing Material

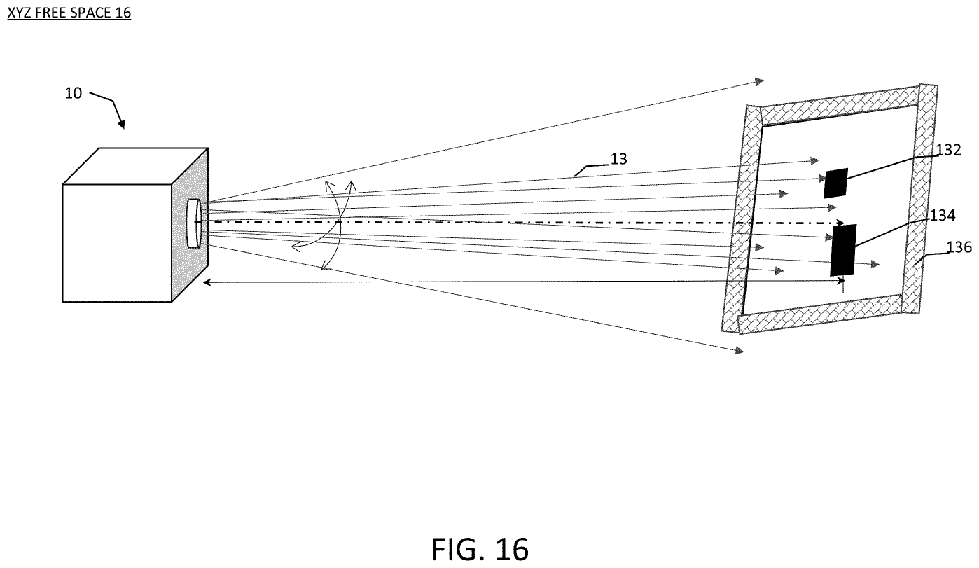

Special material could be placed in the appropriate areas on the walls opposite the laser scanner which absorb the UV light. This would then ensure that no light is scattered or reflected downward towards people. This special material could be decorative in nature and come in a variety of colors and finishes to fit within the design of the room. See diagrammatic depiction in FIG. 16. Light absorbing material are known to those skilled in the art. Such material (e.g., 132 and/or 134) could be created with a form factor needed or desired. They could be positioned on a wall or anywhere in the scanning range of the laser to absorb, and thus, deter reflection or refraction of laser energy to places not desired.

Details about light absorbing materials, including UV laser energy, can be found at U.S. Pat. No. 6,916,866B2 issued Jul. 12, 2005 to Joachimi et al. entitled Laser-absorbing molding compositions with low carbon black contents incorporated by reference herein and U.S. Pat. No. 10,172,705 B2 issued Jan. 8, 2019 to Benz et al. entitled Ultraviolet light absorbing materials for intraocular lens and uses thereof incorporated by reference herein.

As will be appreciated, material 132 or 134 could alternatively be reflective to intentionally redirect laser energy towards a location or away from a location.

(b) Special Targeting Material

Special material could be placed in the appropriate areas on the walls opposite the laser scanner. This special material would be recognized only by a camera system inside the laser scanner which indicates to the laser controller the areas to turn on the laser.

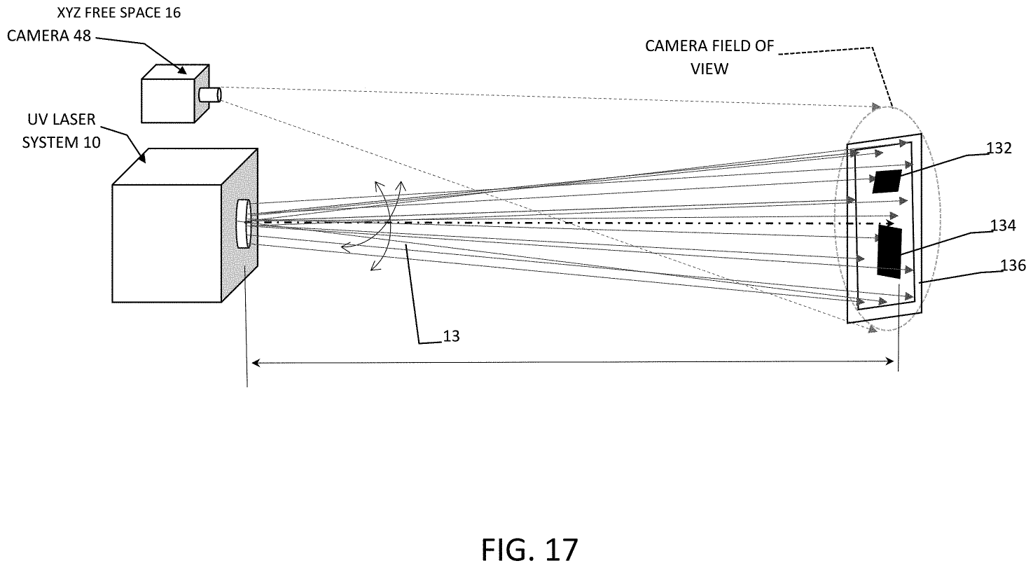

As diagrammatically indicated by the material at border 136 in FIGS. 16 and 17, such material or surface treatment could be recognized by image recognition software which, in turn, would inform the scanning laser controller not to allow laser scanning of such areas or directions. Material 136 forms a rectangular border that could act as an outer boundary within which the laser stays. Alternatively, patches 132 or 134 could designate certain areas that should not be scanned. The materials could be used to tell the controller (via camera or sensor recognition) to stay within the material, stay outside the material, or otherwise.

Details of image recognition of materials can be found at U.S. Pat. No. 9,025,866 issued May 5, 2015 to Liu entitled Material recognition from an image incorporated by reference herein.

Details of image recognition to detect presence and position of light beams can be found at U.S. Pat. No. 8,014,041B2 issued Sep. 6, 2011 to Suzuki et al. entitled Optical scanning apparatus and image forming apparatus, incorporated by reference herein, and US20090195790A1 published Aug. 6, 2009 to Zhu et al. entitled imaging system and method incorporated by reference herein.

As a will be appreciated by those skilled in the art, image recognition can be programmed or trained to identify differences in surfaces or materials in a number of ways. Non-limiting examples are color, surface texture, depth, and reflectivity. Others are possible.

Details about how digital camera or optical sensor image recognition software can be configured and operated for use herein, and machine-learning or deep learning software can likewise, can be found at U.S. Pat. No. 9,436,895B1 issued Sep. 6, 2016 to Jones et al. entitled Method for determining similarity of objects represented in images (Mitsubishi) incorporated by reference herein; U.S. Pat. No. 10,565,433B2 issued Feb. 18, 2020 to Wechsler et al. entitled Age invariant face recognition using convolutional neural networks and set distances, incorporated by reference herein; and U.S. Pat. No. 9,602,783B2 issued Mar. 21, 2017 to Sugishita, et al, entitled image recognition method and camera system, incorporated by reference herein.

Benefits of Laser Based Upper-Room UVGI

Faster-Farther-Safer summarize the main benefits which stem from the laser scanner based UVGI approach instead of the more common lan1p-based approach:

Faster—Controllability & Efficiency
a. A single laser can scan sweep across an entire room and disinfect it much faster than lamp-based approaches.
b. The lasers are the most efficient way to send the light only where you want it and no other places. Historically Upper-Room UVGI has suffered in implementations since you have to insure the people in the room are not exposed to the UV light-hard to do with UV lamps. Easily done with an eye-safe invisible laser.
c. The laser-based approach will be modular, is easy to install and can conform to any shape room (it does not need to be rectangular).
d. The UV laser consumes <20 W of standard US wall plug electrical power whereas most mercury lamps in the UVGI studies consumed 50-300 W of power.

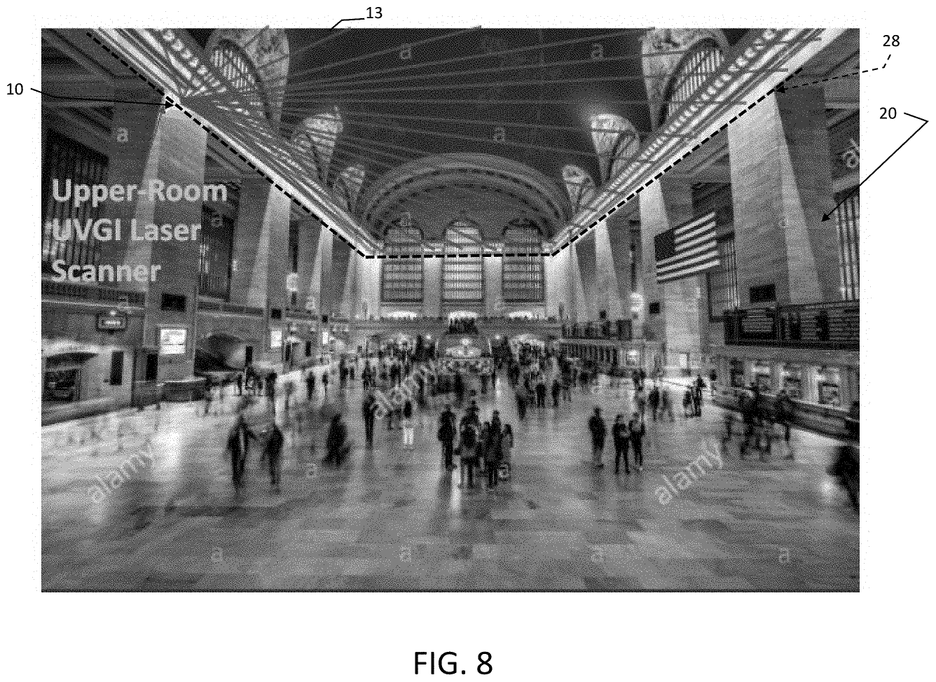

Farther
a. FIG. 8 shows how a Laser based Upper-Room UVGI could be deployed in New York City's Grand Central Station. A single laser 12 or laser system 10 could perform a single scan of the main concourse (35,000 ft²) in 30 s, and could be controlled directionally to scan above plane 28 only for safety. For this case multiple lasers and/or laser system 10 would be recommended.

Safer
a. Lasers can operate with extreme precision. It's very easy to minimize people's exposure to a laser beam. Commercial laser light shows do this daily. During installation, personnel will ensure the beam does not reflect off any reflective surfaces which would send too much power downward towards people.
b. Depending on features in the room, the scanning controller can be programmed to turn off the beam in certain areas where people would be near the ceiling (e.g., staircases, escalators, etc.).
c. If use in proximity to people is impossible, night cleaning can still be very effective. Initially, operators could be ‘in the loop’ until all personnel are comfortable with the system. Likely, they will be more afraid of COVID-19 than the lasers which kill it.
d. Alakai's Patented Stimulated Aversion technique can be deployed for eye-safety. That U.S. Pat. No. 8,724,097 integrates a non-harmful light source with the UV laser. The non-harmful light source (e.g., green light) is of should intensity that a human viewer would experience disability or annoyance glare and immediately avert their eyes. Thus, even if they have direct line of site of the visible/UV combined light energy, they would be induced to avert direct view and thus reduce chance of eye injury. The patent describes how to combine the visible and UV beams into one and is incorporated by reference herein.

261 nm UV Laser 310 (Smaller than a Stick of Butter) (FIG. 9)

As mentioned, embodiments of the invention could benefit from UV laser 310 of FIG. 9. A beneficial technology that can assist in transforming the “UV lamp” old approach is such a new, low-cost, small deep ultraviolet laser. This laser was developed to insert into a DARPA system. The laser functions at 125 mW CW.

In one example, UV laser 130 of FIG. 9 has the following operating characteristics:
Wavelength: 261 nm
Output power: 30-150 mW, continuous wave
Output stability: <5%
Output beam size: circular 0.5-3 mm with <3 mrad divergence
Input power: +12 VDC.

As can be seen, system 10 can be easily and efficiently combined so that the scanning and control electronics are a single box or housing 11. A small camera 48 and AI control module could provide advanced functionality of the type discussed above. This would include Deep Learning algorithms that could easily detect personnel that were close to the beam and shut down the laser and other safety interlocks. In addition, these advanced control features could detect when nobody was in the room and then have the laser scan the lower part of the room thereby sterilizing the other surfaces which are exposed.

### 2. Specific Example Embodiment 2: UVGI on Existing Vents

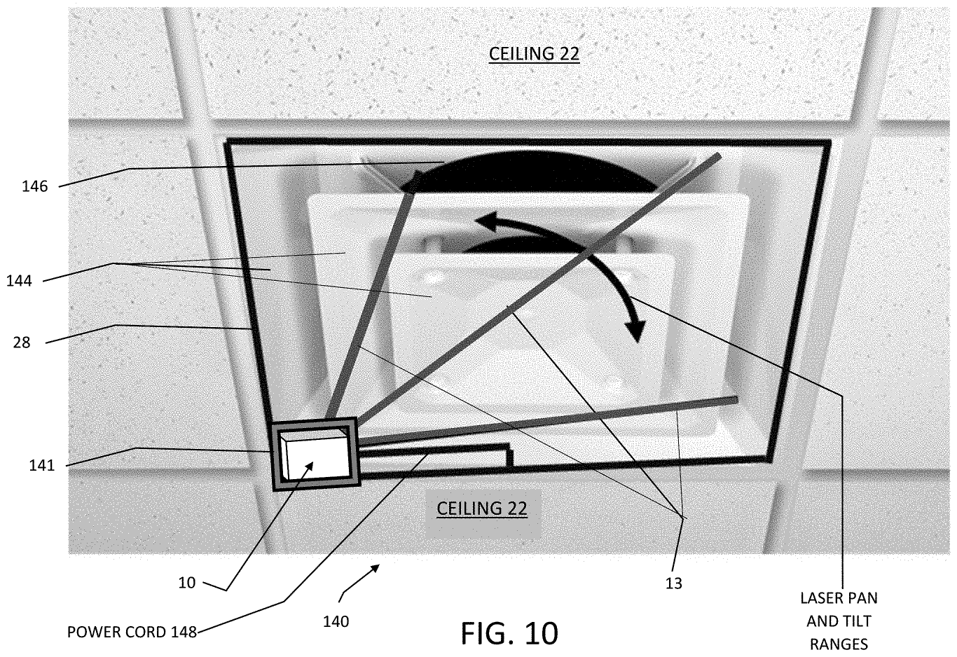

With particular reference to FIGS. 10-12, aspects of the invention can be implemented in similar fashion to the upper-room embodiment in the following ways.

For some application it may be more desirable to add the UV laser scanner 10 onto an existing air vent (see, e.g., vent 140 in room ceiling 22) (either intake or outlet) as shown in FIG. 10. In this case the UV laser would not be scanning the entire room but just the area directly under the vent. In this case, the laser power density could be increased to scan the air flowing through vent opening(s) 146 it faster and since there is always a direct backstop on the system there are minimal eye-safety concerns.

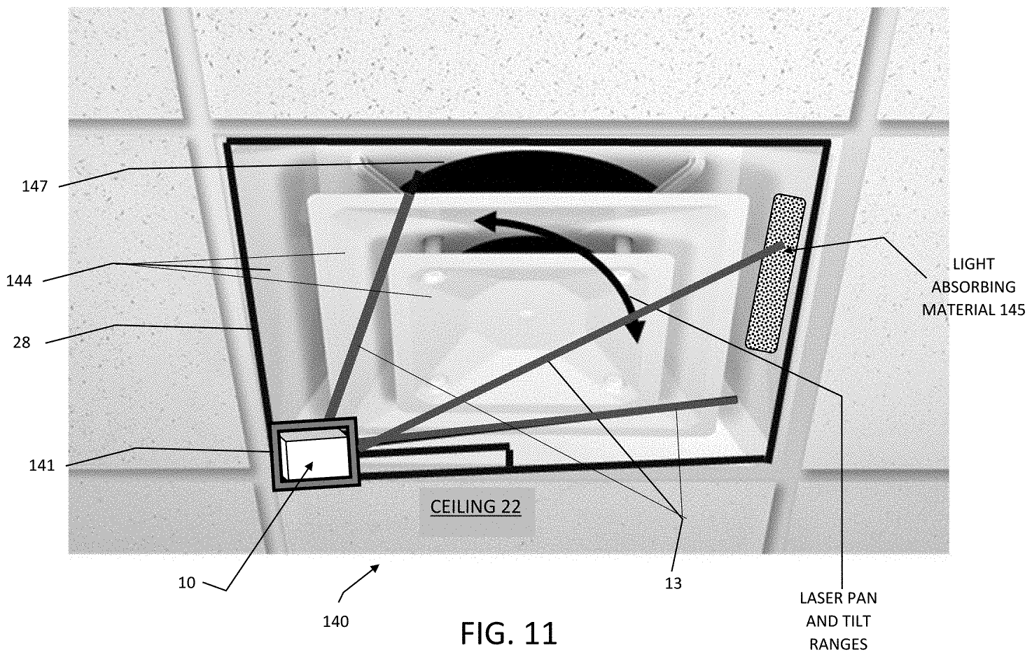

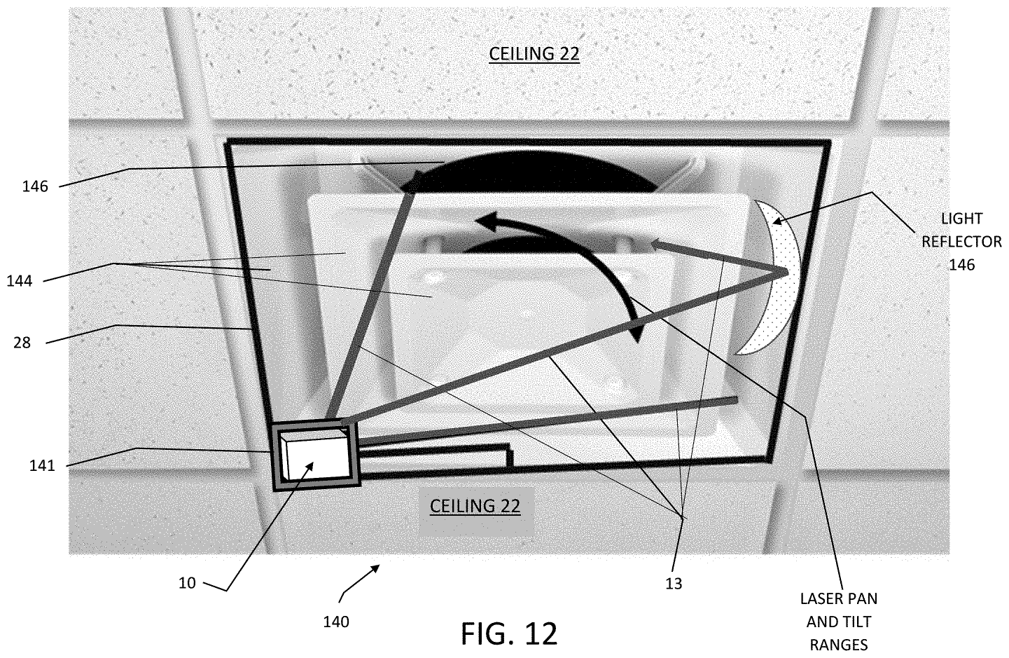

Laser scanner 10 can use the operating principles of Specific Example 1 and have analogous laser control electronics. It could be mounted by an appropriate mounting bracket 141 to make it retrofittable and removeable for repair or replacement. Fastening techniques can vary. Non-limited examples are clamps, screws, bolts, adhesives, and interference fit. Electrical power can be via hardwire 148 or battery or other.
a. Laser 13 can be directed over pan/tilt scanning limits so as to not leave the vent 140. (LEDs/lamps make stray light control more difficult/impossible). It can be programmed to stay within perimeter 28.
b. Vent sides or surfaces 144 may be absorptive or reflective in whole or in part (e.g., with added material with appropriate properties). See, e.g., FIG. 11 at location 145.
c. Sides 144 may be curved to better contain laser radiation, and maximize use of power. Alternatively, a reflector 146 could be added to redirect laser energy as needed or desired. See, e.g., FIG. 12 at 146.
d. Easily replaceable.
e. May use optics or a scanning system to cover cross-sectional area.
f. Can fit to standard vent sizes, or custom sized.

### 3. Specific Example Embodiment 3: Human-Employed Handheld UVGI Laser Scanner

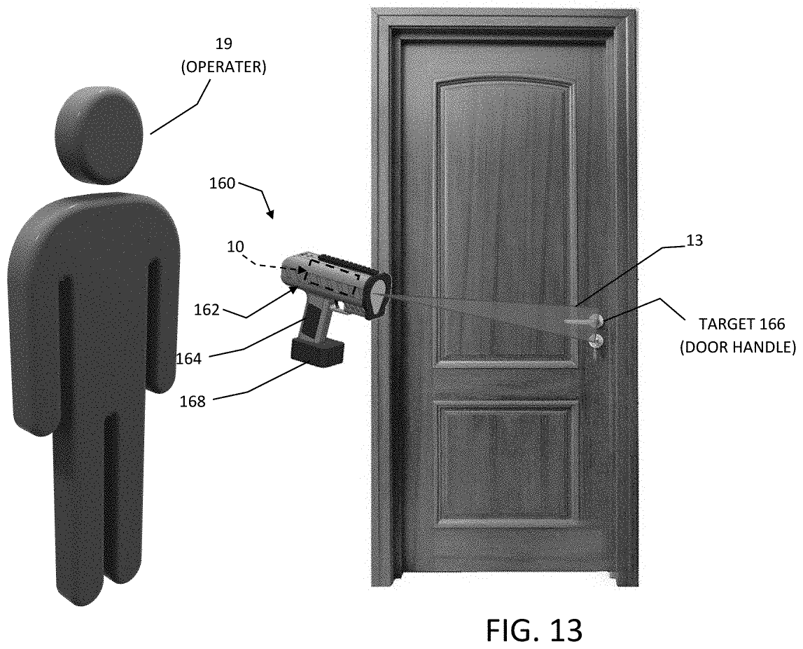

FIG. 13 illustrates another possible implementation/embodiment according to aspects of the invention. A laser system 10 could be housing inside body 162 of hand-held device 160. A user 19 simply holds handle 164 and points and shoots the laser beam 13 to the intended target 166 (here a frequently used door handle). Unit 160 can have an on-board battery power source 168 for completely portable operation.

UVGI could also be implemented with a handheld system 160 as shown in FIG. 13. In this case the system would be flood an area or surface (either by a scanning mirror or a set of refractive/reflective optics) and the operator would expose the area for a specified amount of time (a few seconds). In addition to the concept below, the UVGI scanning sub-system could be applied as an add-on module to the UV Raman sensor described in Applicant's patent application (US 20190109431 A1), incorporated by reference herein. As such, combining the UV scanning laser with that of US 20190109431 would allow handheld unit 160 to both interrogate a target 166 for presence of a chemical species of interest (e.g., explosive, poison, etc.), by also sterilize target 166.

Details about a hand-held Raman laser-based system can be found at US 2019/0109431 A1, published Apr. 11, 2019 to Waterbury et al. entitled UV lasers and UV Raman System for effective and efficient molecular species identification with Raman Spectroscopy incorporated by reference herein.

A cleaning crew carrying the right form of laser scanner could disinfect small or large areas faster, safer, and without expendable cleaning supplies. Neither cleaning fluids, nor paper products, which are hard to come by since the start of the novel corona virus pandemic in 2021 are needed.

### 4. Specific Embodiment 4: Autonomous UVGI

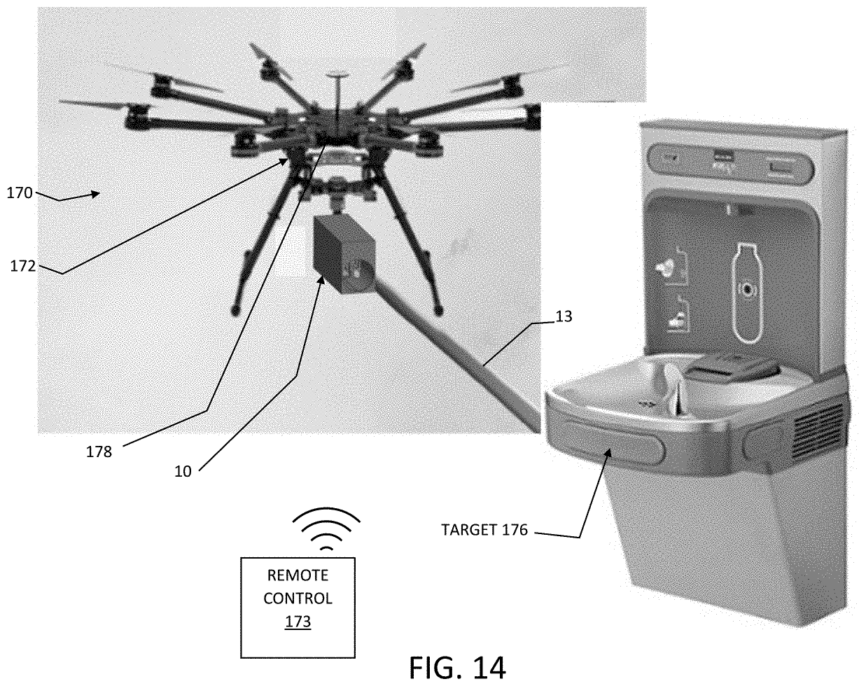

FIG. 14 illustrates a still further embodiment according to aspects of the invention. Here a robot or remote-controlled unmanned vehicle 170 can carry on its frame 172 a laser scanning system 10 and an on-board battery 178 to power it.

In this case the UV scanning system could be placed on an autonomous vehicle (either ground robot or air robot). The robotic platform could contain a camera and then using AI and automatic target recognition algorithms the robot could survey an area automatically and sterilize these areas. The camera-based system would also contain the facial recognition safety sub-systems previously described to ensure complete system safety.

The unattended Air vehicle robot 170 carries the UV laser scanner 10. It likely would include a camera 48 and/or camera module 80 or other sensor to assist in navigation and/or fining the target. Remote control 173 could allow human control or monitoring of operation and could have a video display of the camera field of view, as is well known with drones and other robotic platforms. In this case the camera system is in the UV laser scanner and would ensure the entire area of the desired target 176 (water fountain in this case) is scanned and sterilized.

Robotic UVGI Laser Scanner. Once developed this UVGI Laser Scanner could be deployed on robotic platforms to autonomously go through an area scanning all surfaces of interest.

Details of robotic movement and control can be found at U.S. Ser. No. 10/846,873B2 issued Nov. 24, 2020 to Versace et al. entitled Methods and apparatus for autonomous robotic control, incorporated by reference herein.

D. Options and Alternatives

As will be appreciated by those skilled in this field, the invention can take many forms and embodiments. Some of those variations are mentioned herein. Other examples are:

1. UV Laser Light Source.

US Patent Publication 2019/0109431, owned by Applicant, and which is incorporated by reference here, discusses one possible example and operating principles. Others are possible.

2. Control of Laser Light Source.

US Patent Publication 2019/0109431 and U.S. Pat. No. 8,724,097 owned by Applicant and incorporated by reference herein, discuss at least some of the operating parameters and ways to control them. This includes aiming, scanning, controlling power through generation or duty cycle, etc. Others are possible. U.S. Pat. No. 8,724,097 also discussed human safety techniques regarding lasers in the presence of humans.

3. Other.

US Patent Publication 2019/0109431 and U.S. Pat. No. 8,724,097 which are incorporated by reference here discuss at least some of the operating parameters and ways to control them, including in the context of using UV lasers in Raman spectroscopy sensors. In that mode, the UV laser is focused on or scanned across a target to excite Raman scattering. Collection of the scattering can be processed to try to detect the presence of a chemical species at the target. As will be appreciated, that mode could optionally be integrated with use of the UV laser for UVGI in a UVGI mode. The same system then both try to identify chemical information about a space or area (such as to identify substances most likely to collect or attract a pathogen such as a virus), and then automatically or semi-automatically use UVGI mode to direct UV light on that identified area.

Other options and alternatives are, of course, possible.

The required power level for viral disinfection has been demonstrated in excess of what is needed to do the job with plenty of design margin.

The eye-safety technique and subsystem has been well vetted by numerous organizations, especially US Army Public Health Command (USAPHC). The CDC has published guidelines for Upper-Room UVGI and the embodiments herein would follow those guidelines as appropriate.

What is claimed is:

1. A method of sterilizing or disinfecting a space or surface comprising:
a. generating UVGI light from a laser;
b. directing the laser into a space or area to be disinfected or sterilized; and
c. operating the directed laser for a time and power effective for UVGI in the space or at the area.

2. The method of claim 1 further comprising controlling one or more of the following effective for human safety in the presence of the laser:
a. power; and
b. direction.

3. The method of claim 2 wherein the laser is controlled for scanning a space or area.

4. The method of claim 2 wherein the laser is controlled to avoid humans.

5. The method of claim 4 wherein the avoiding of humans comprises directing the laser in a room above standing height of humans.

6. The method of claim 1 wherein the directing the laser comprises scanning the laser across a space or area.

7. The method of claim 1 in combination with using the UV laser in a UVGI mode and in a UV Raman spectrometer mode.

8. The method of claim 1 wherein the directing can be by hand-held unit.

9. The method of claim 1 wherein the directing can be via an unmanned automatous vehicle.

10. The method of claim 1 in combination with an interior room having the presence of humans at certain times, wherein the method adjusts dynamically to sensing indicative of presence of humans including eye and skin safety or eye aversion techniques.

11. A system of method of sterilizing or disinfecting a space or surface comprising:
a. a UV laser generating UVGI;
b. means to direct the UV laser into a space or area to be disinfected or sterilized; and
c. means to control the directed laser for a time and power effective for UVGI in the space or at the area.

12. The system of claim 11 wherein the means to direct comprises one of:
a. a manually aimable UV laser housing; or
b. an electro-mechanically aimable actuator.

13. The system of claim 11 wherein the means to control comprises one of:
a. manually adjustable controls; or
b. automatically adjustable controls.

14. The system of claim 11 in combination with a UV Raman spectrometer sensor.

15. The system of claim 11 in combination with a digital camera or imager including image processing software.

16. The system of claim 11 wherein laser control including one or more of:
a. control of laser direction;
b. control of laser scanning speed;
c. control of laser power density;
d. control of laser beam size;
e. control of laser beam spread;
f. inclusion of eye aversion;
g. direction restriction;
h. control of laser on and off based on a sensed parameter;
i. control of laser on and off based on a time of day regimen; and/or
j. control of laser absorption or reflection.

17. The system of claim 11 mounted on an unmanned vehicle.

18. The system of claim 11 mounted in a portable handheld unit.

19. The system of claim 11 mounted at or near an HVAC room vent or air flow outlet or return.

20. The system of claim 11 wherein the laser is controlled relative to a room to be limited to upper-room laser scanning.
